(19) United States
(12) Patent Application Publication
(10) Pub. No.: US 2014/0066782 A1
(43) Pub. Date: Mar. 6, 2014

(54) SYSTEM AND METHOD FOR DETERMINING A RESTING HEART RATE OF AN INDIVIDUAL

(71) Applicants: Paul Stanley Addison, Edinburgh (GB); Rui Wang, Livingston (GB); James Nicholas Watson, Dunfermline (GB)

(72) Inventors: Paul Stanley Addison, Edinburgh (GB); Rui Wang, Livingston (GB); James Nicholas Watson, Dunfermline (GB)

(73) Assignee: Nellcor Puritan Bennett LLC, Boulder, CO (US)

(21) Appl. No.: 13/598,953

(22) Filed: Aug. 30, 2012

(51) Int. Cl. A61B 5/024

(52) U.S. Cl. 600/476

(57) ABSTRACT

A system to determine a resting heart rate (HR) of an individual. The system may include a monitor that is configured to be operatively connected to a sensor that obtains physiological signals from an individual. The monitor is configured to receive the physiological signals from the sensor. The monitor may include a validation module that is configured to analyze the physiological signals to identify valid heart beats from the physiological signals. The monitor may also include a rate-determining module that is configured to determine an HR signal that is based on the valid heart beats. The HR signal includes a series of data points. The monitor may also include an analysis module that is configured to analyze the HR signal and identify baseline data points from the series of data points. The analysis module is configured to calculate the resting HR based on the baseline data points.

### FIELD

Embodiments of the present disclosure generally relate to physiological signal processing and, more particularly, to processing physiological signals to determine a heart rate.

### BACKGROUND

Heart rate is a physiological parameter that is frequently used by doctors or other medical professionals to diagnose and/or monitor a medical condition of a patient. An individual's heart rate (i.e., a number of heart beats per unit of time) may be extracted from a variety of physiological signals using various types of monitoring systems. For example, an electrocardiograph (ECG) detects electrical activity that corresponds to muscle excitation of the heart. A pulse oximeter (or photoplethysmograph (PPG)) measures changes in light absorption in which the changes are indicative of blood flow through an anatomical part (e.g., a finger). A phonocardiograph (PCG) is another type of monitoring system that detects sounds caused by the closing of heart valves. In each of the above monitoring systems, oscillating waveforms may be detected that correspond to heart beats. A heart rate may be determined by, for example, counting the number of heart beats in a set period of time and dividing the number by that time.

A resting heart rate may represent the heart rate of an individual when he or she is inactive (i.e., when resting). The resting heart rate may be used with respect to diagnosing and/or monitoring a medical condition or physiological event. For example, a significant association exists between resting heart rate and cardiovascular mortality. One method of determining a resting heart rate includes counting a number of heart beats over an extended period of time and determining the resting heart rate from the number of heart beats. However, resting heart rates can vary for different reasons and the variability may affect the estimated resting heart rate. For example, during the period of time that the heat beats are counted, the heart rate may momentarily rise due to short term activity or other events (e.g., movement by the patient, excitement, response to pain, and the like). If the heart rate momentarily rises due to such activity or events, the estimated resting heart rate will be above a truer resting heart rate for the individual. Additionally, the variability of the detected heart rate may differ based on the method of detection. For example, a heart rate detected by a pulse oximeter may vary more than a heart rate detected by an ECG. Such variability may affect the estimated resting heart rate.

### SUMMARY

Certain embodiments provide a system for determining a resting heart rate (HR) of an individual. The system may include a monitor that is configured to be operatively connected to a sensor that obtains physiological signals from an individual. The monitor is configured to receive the physiological signals from the sensor. The monitor may include a validation module that is configured to analyze the physiological signals to identify valid heart beats from the physiological signals. The monitor may also include a rate-determining module that is configured to determine an HR signal that is based on the valid heart beats. The HR signal includes a series of data points. The monitor may also include an analysis module that is configured to analyze the HR signal and identify baseline data points from the series of data points. The analysis module is configured to calculate the resting HR based on the baseline data points.

In some embodiments, the analysis module is configured to obtain a first derivative of the HR signal and establish a derivative threshold value. The first derivative includes derivative data points, wherein the analysis module identifies derivative data points of the first derivative that are below the derivative threshold value. The baseline data points of the HR signal correspond to the derivative data points identified by the analysis module.

The analysis module may also be configured to obtain a first derivative of the HR signal and establish a designated range. The analysis module identifies derivative data points of the first derivative that are within the designated range, wherein the baseline data points of the HR signal correspond to the derivative data points identified by the analysis module

In some embodiments, the baseline data points have HR values that are less than a designated HR threshold value. In some embodiments, the HR signal may form a HR waveform. The analysis module may analyze a contour of the HR waveform to identify a spike in the HR waveform. The spike includes non-baseline data points. The non-baseline data points may be excluded from the calculation of the resting HR.

The resting HR may be a dynamic resting HR that changes over an extended period of time. The analysis module may calculate a baseline trend, wherein the baseline trend is based on the baseline data points and represents the resting HR as a continuous waveform over the extended period of time.

In certain embodiments, the physiological signals include photoplethysmogram (PPG) signals and the monitor includes a PPG monitor. Certain embodiments may include the sensor and the sensor may be a pulse oximetry sensor.

Certain embodiments provide a method for determining a resting HR of an individual. The method may include acquiring physiological signals of the individual from a sensor. The method may also include analyzing the physiological signals to identify valid heart beats from the physiological signals and determining an HR signal based on the valid heart beats. The HR signal includes a series of data points. The method may also include analyzing the HR signal to identify baseline data points from the series of data points and calculating the resting HR based on the baseline data points.

Certain embodiments provide a tangible and non-transitory computer readable medium that includes one or more sets of instructions configured to direct a monitor to acquire physiological signals of the individual from a sensor. The computer readable medium is also configured to analyze the physiological signals to identify valid heart beats from the physiological signals and determine an HR signal based on the valid heart beats. The HR signal includes a series of data points obtained. The computer readable medium is also configured to analyze the HR signal to identify baseline data points from the series of data points and calculate the resting HR based on the baseline data points.

One or more embodiments of the present disclosure may allow for quick and simple determination of a resting HR through analysis of physiological signals, such as ECG signals, PPG signals, and PCG signals, in contrast to previous systems and methods, which typically determine the resting HR based on data that includes aberrations caused by, for example, short term activity, excitement, or response to pain. Moreover, embodiments may be used to calculate a resting HR with a pulse oximetry sensor.

Embodiments may also be used to calculate other physiological parameters in a more reliable manner. Such physiological parameters may include stroke volume and cardiac output. Furthermore, in determining changes in respiratory effort computed from a PPG signal, a baseline shift in the resting HR may be used as an indication of a change in the respiratory effort. Embodiments may also be used to indicate pain and/or to determine long term changes in the resting HR during a therapeutic intervention.

Certain embodiments may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

### BRIEF DESCRIPTION OF THE DRAWINGS

FIG. 1A illustrates a simplified block diagram of a system configured to determine a resting heart rate (HR) of an individual, according to an embodiment.

FIG. 1B illustrates an electrocardiograph (ECG) waveform that may be used to determine the resting HR, according to an embodiment.

FIG. 1C illustrates an phonocardiograph (PCG) waveform that may be used to determine the resting HR, according to an embodiment.

FIG. 1D illustrates a photoplethysmograph (PPG) waveform that may be used to determine the resting HR, according to an embodiment.

FIG. 2 illustrates a HR waveform indicating a HR signal over time and also illustrates a first derivative of the HR waveform, according to an embodiment.

FIG. 3 illustrates an enlarged portion of the first derivative waveform shown in FIG. 2, according to an embodiment.

FIG. 4 illustrates another HR waveform indicating a HR signal over time, according to an embodiment.

FIG. 5 illustrates a flow chart of a method of determining a resting HR, according to an embodiment.

FIG. 6 illustrates an isometric view of a PPG system, according to an embodiment.

FIG. 7 illustrates a simplified block diagram of a PPG system, according to an embodiment.

### DETAILED DESCRIPTION

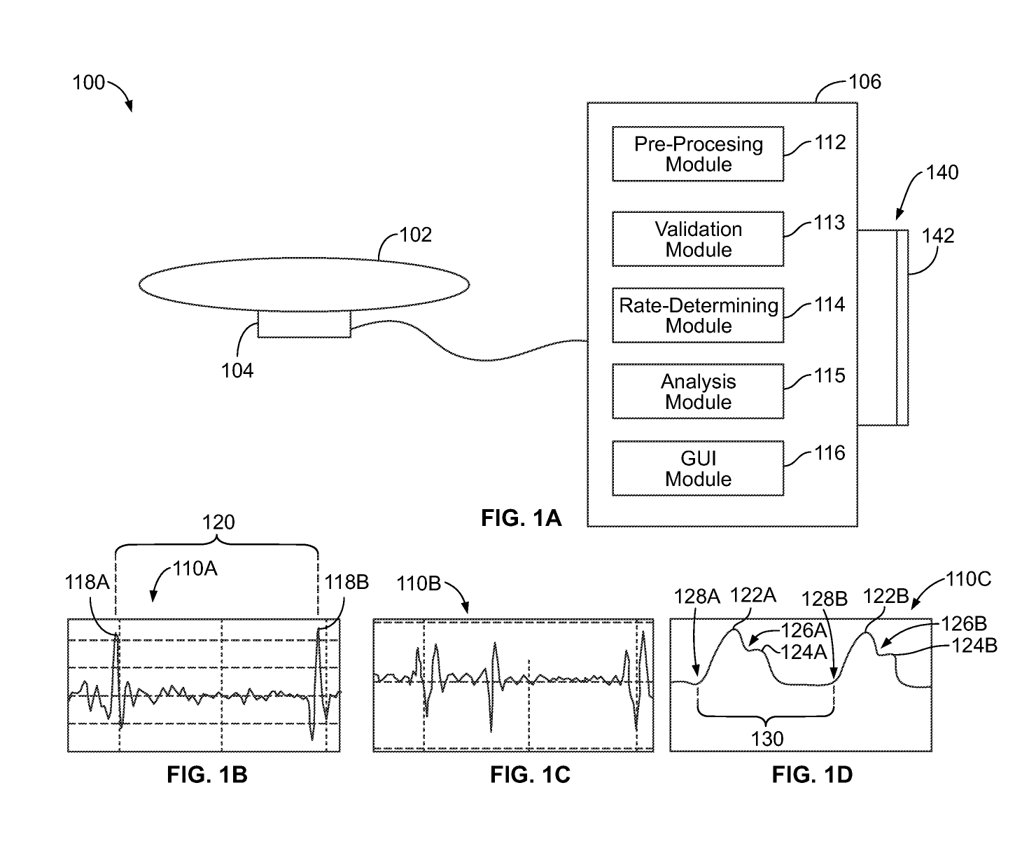

FIG. 1A illustrates a simplified block diagram of a system 100 configured to determine a resting heart rate (HR) of an individual. The system 100 is configured to acquire physiological signals (or biosignals) from an individual (e.g., patient) 102 and analyze the physiological signals to determine a physiological parameter, such as a resting HR. The physiological signals are indicative of phenomena occurring in the patient. For example, the physiological signals may describe cardiac activity in which the heart undergoes a number of cardiac cycles (e.g., heart beats). The physiological signals may be electrical, optical, and/or acoustical signals.

The system 100 may include a sensor 104 that is configured to detect one or more types of physiological signals. By way of example, the system 100 may include an electrocardiograph (ECG) system that detects electrical signals corresponding to muscle excitation of the heart. In such cases, the sensor 104 may include a plurality of electrodes that are coupled to different anatomical locations of the individual 102 (e.g., chest, wrists, and/or ankles). FIG. 1B illustrates, according to an embodiment, a representative ECG waveform 110A based on the ECG signals acquired by the electrode-sensor 104. As another example, the system 100 may include a phonocardiograph (PCG) system that detects sounds that may be caused by the closing of heart valves. In such cases, the sensor 104 may include one or more microphones that are coupled to the individual 102. FIG. 1C illustrates, according to an embodiment, a representative PCG waveform 110B based on the PCG signals acquired by the microphone-sensor 104. In alternative embodiments, the system 100 includes an ultrasound system configured to detect heart beats from the individual 102.

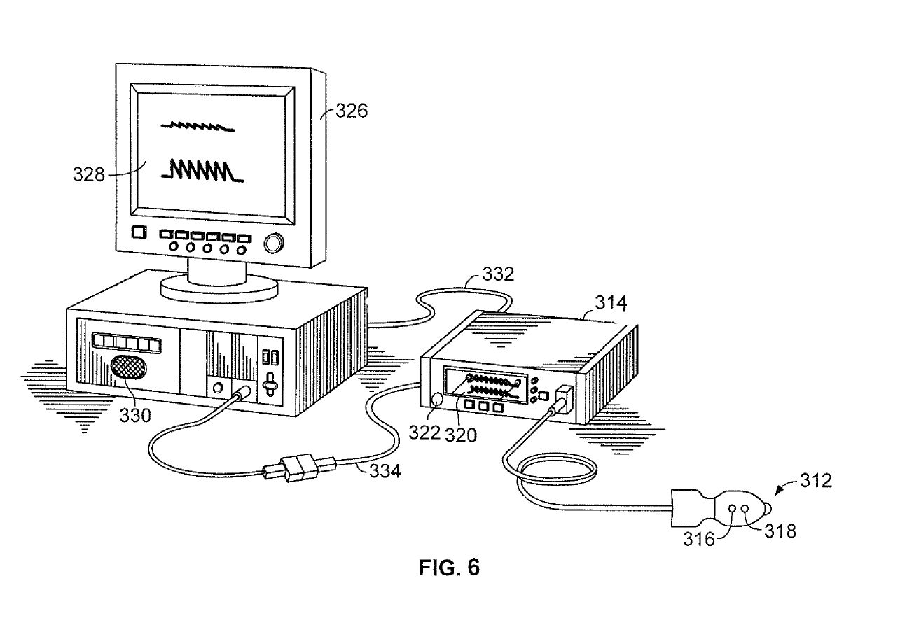

In certain embodiments, the system 100 includes a photoplethysmograph (PPG) system, which can measure changes in blood volume through an anatomical portion or location (e.g., a finger). A typical example of a PPG system is a pulse oximetry system, such as the pulse oximetry system 310 shown in FIG. 6, although other PPG systems exist and may be used with embodiments described herein. The PPG-sensor 104 may include a probe having one or more light sources and one or more light detectors that are coupled to the individual 102. The light source(s) provide an incident light that is scattered, absorbed, reflected, and/or transmitted by the blood. The light detector(s) detect an amount of light that may be correspond to blood volume. For example, as the volume of blood increases at the anatomical location, the light is attenuated more and, as such, a smaller amount of light is detected. FIG. 1C illustrates, according to an embodiment, a representative PPG waveform 110C based on the PPG signals acquired by the PPG-sensor 104.

As shown in FIG. 1A, the system 100 may include a monitor (or computing system) 106 that includes one or more components for analyzing and/or processing the physiological signals. For example, the monitor 106 may include a pre-processing module 112, a validation module 113, a rate-determining module 114, an analysis module 115, and a graphical user interface (GUI) module 116. As used herein, a “module” may include hardware components (e.g., processor, controller), software components, or a combination thereof including any associated circuitry.

The pre-processing module 112 is configured to remove unwanted signal data (e.g., noise) from raw physiological signal data obtained from the individual 102. For example, raw PPG signals may include artifacts caused by motion of the patient relative to the light detector, instrumentation bias (e.g., bias by amplifiers used in the PPG system), powerline interference, low amplitude PPG signals, etc. Raw physiological signals from other types of monitoring systems, such as ECG and PCG systems, may also include unwanted noise. The pre-processing module 112 is configured to remove the noise to provide clearer and/or cleaner physiological signals to the other components of the system 100.

The validation module 113 is configured to analyze the physiological signals to identify valid heart beats from the physiological signals. In some embodiments, the validation module 113 is part of the pre-processing module 112 or the rate-determining module 114. The validation module 113 may analyze the physiological signals after the physiological signals have been processed as described above. In some embodiments, the validation module 113 examines the physiological signals to identify one or more reference features in the physiological signals. For instance, a series of data points over time may provide waveforms, such as the waveforms 110A-110C. A reference feature may be an identifiable point, segment, or characteristic of the waveform (e.g., peak, trough (or foot), notch, slope of a designated segment, threshold, etc.) that may be relied upon in analysis of the physiological signals. In many cases, a reference feature of a waveform corresponds to a known physiological activity (e.g., excitation of heart muscles, closure or opening of valves, maximum volume of blood at an anatomical location, etc.). The validation module 113 may examine the data points, or a select number of data points (e.g., a segment of the waveform), to confirm that the data points are caused by a designated event of a cardiac cycle and are not a result of noise or other unwanted event, such as when the sensor 104 is being adjusted. The data points associated with valid heart beats may then be used by the rate-determining module 114 to determine a HR signal. In some embodiments, the data points that are not identified as corresponding to heart beats may not be considered in subsequent analysis.

The rate-determining module 114 is configured to analyze the heart beats or, more specifically, the data points corresponding to the valid heart beats identified by the validation module 113 and determine a HR of the individual at a designated moment of time. For example, the HR may be calculated by analyzing time intervals between two or more heart beats or by analyzing portions of a waveform that corresponds to a single heart beat. By way of example only, when analyzing the physiological signals, the rate-determining module 114 may identify one or more reference features (e.g., points, segments, and/or characteristics that correspond to a waveform) that may be used to calculate HR. For example, in the ECG waveform 110A, the rate-determining module 114 may identify an R-wave peak 118 in each heart beat. A time interval 120 between the R-wave peaks 118A, 118B may be determined and its inverse used to calculate the HR. For example, if the time interval is 0.90 seconds between the two R-wave peaks 118A, 118B, then the HR is 67 beats/minute.

Corresponding to each heart beat, the PPG waveform 110C may include a systolic peak 122, a diastolic peak 124, and a dicrotic notch 126 that exists therebetween. In some cases, the diastolic peak is not a peak but instead a change in slope. To determine HR, the rate-determining module 114 may identify for each heart beat a reference point that exists at a foot 128 of the wave before the systolic peak 122. The HR may be determined in a similar manner as described above with respect to the ECG waveform by identifying a time interval 130 between the foot 128A and the foot 128B.

However, it should be noted that the above description is just exemplary and that many reference points and/or waveform segments may be analyzed and used in calculating a HR of an individual. Furthermore, the physiological signals may be processed in various manners to determine a HR. For example, a first derivative or second derivative of the PPG waveform may be used to locate certain reference data points in the PPG waveform.

As will be described in greater detail below, the analysis module 115 is configured to identify baseline data points from the physiological signals. The baseline signal data points may be a limited number of data points from a series of data points that also include non-baseline data points. The baseline data points may correspond to a time when the individual is inactive (e.g., at rest). The non-baseline data points may correspond to short term activity by the individual or another event. For example, the non-baseline data points may correspond to moments or time periods when the patient is moving, excited, responding to pain, and the like. To calculate a resting HR, the analysis module 115 may include the baseline data points. The analysis module 115 may also exclude the non-baseline data points in calculating the resting HR.

The system 100 may also include a user interface 140 that includes a display 142. The user interface 140 may include hardware, firmware, software, or a combination thereof that enables a user to directly or indirectly control operation of the system 100 and the various components thereof. The display 142 is configured to display one or more images, such as one or more of the waveforms 110A-110C. The display 142 may also be configured to show the current HR (not shown) and the resting HR (not shown). In some embodiments, the user interface 140 may also include one or more input devices (not shown), such as a physical keyboard, mouse, touchpad, and/or touch-sensitive display. The user interface 140 may be operatively connected to the GUI module 116 and receive instructions from the GUI module 116 to display designated images on the display 142. The user interface 140 may also include a printer or other device for providing (e.g. printing) a report.

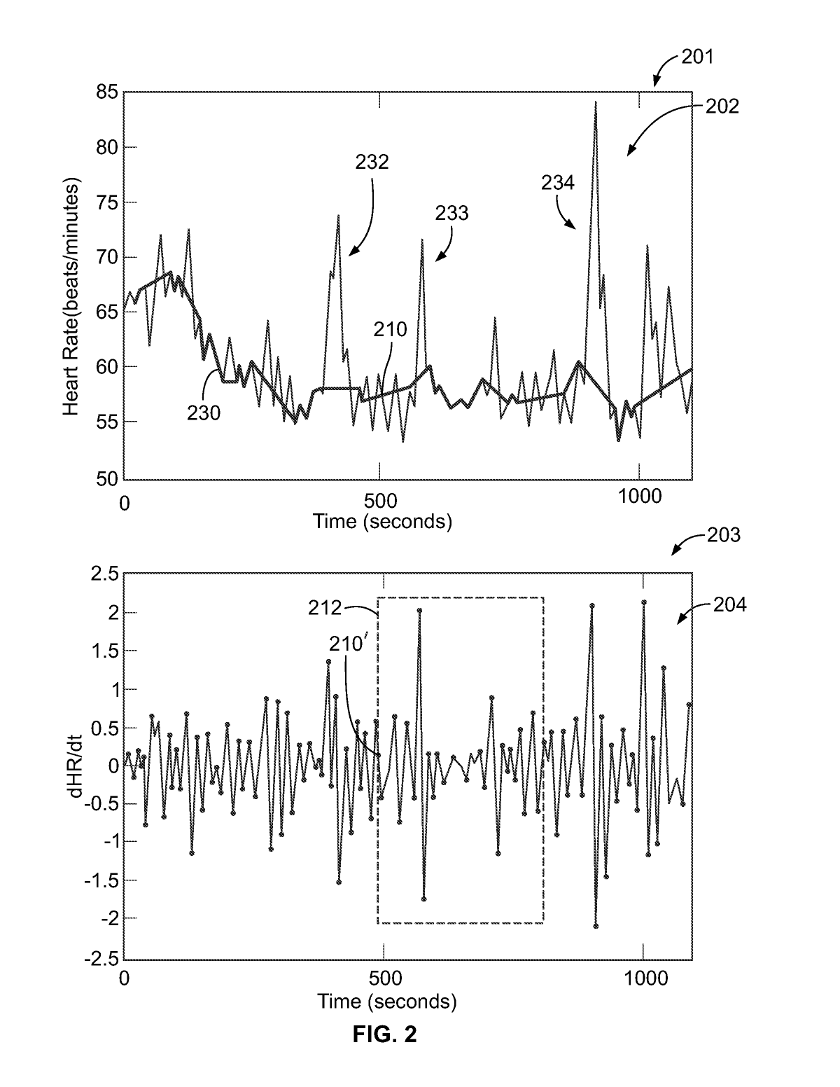

FIG. 2 illustrates a HR signal graph 201 that includes an HR waveform 202 showing an HR signal over time. FIG. 2 also illustrates a first derivative graph 203 that includes a first derivative waveform 204. The HR signal graph 201 and the first derivative graph 203 are positioned in FIG. 2 so that equal time points are vertically aligned (e.g., 500 seconds for the HR signal graph 201 is vertically aligned with 500 seconds of the first derivative graph 203). The HR waveform 202 may be generated by the rate-determining module 114 (FIG. 1A). The HR waveform 202 includes a series of data points in which each data point may correspond to a calculated HR value at a designated moment in time. A series of data points in the HR signal may correspond to a segment of the HR waveform 202. As discussed above, embodiments described herein may identify baseline and/or non-baseline data points. Likewise, embodiments described herein may identify baseline segments, which may include a series of baseline data points, and non-baseline segments, which may include a series of non-baseline data points.

The first derivative waveform 204 corresponds to a first derivative of the HR signal (dHR/dt) and may be generated by the analysis module 115 (FIG. 1A). Each data point of the first derivative waveform 204 indicates a rate of change of the HR signal at a designated moment in time. As such, the slope of the HR waveform 202 is zero when the first derivative waveform 204 is zero (i.e., when the first derivative waveform 204 intersects a horizontal axis at dHR/dt=0). By way of example, a data point 210 at approximately t=500 seconds of the HR waveform 202 may have an HR value of about 58 heart beats/minute and also correspond to a local peak of the HR waveform 202. A corresponding data point 210′ of the first derivative waveform 204 corresponding to approximately t=500 seconds is about equal to zero because the rate of change at the local peak of the HR waveform 202 is zero.

In the illustrated embodiment, the HR waveform 202 is shown as heart beats/minute, but other HR units may be used. For example, the HR signal may correspond to beat-to-beat time intervals (e.g., a time interval between first and second R-wave peaks in an ECG or a time interval between first and second troughs in a PPG). As such, instead of being (events)/(unit of time), the HR signal may simply be a unit of time (e.g., seconds or milliseconds).

Embodiments described herein may analyze and process the HR signal extending over a designated period of time. The designated period of time may also be referred to as a time window. For example, the time window may be at least 10 seconds, 20 seconds, 30 seconds, 45 seconds, 60 seconds, two minutes, five minutes, ten minutes, thirty minutes, or more. An exemplary time window 212 is shown in FIG. 2 and extends between about t=500 seconds to about t=800 seconds. For each time window, embodiments described herein may identify baseline data points within the time window and calculate a resting HR for the time window that is based on the baseline data points. The non-baseline data points may be excluded in the calculation or weighted in a manner that reduces the significance of the non-baseline data points.

As one example, the resting HR may be an average of the HR values of the baseline data points in a corresponding time window. For instance, if 30 data points exist within a time window and 25 of the 30 data points are identified as baseline data points, the resting HR may be a sum of each of the HR values of the baseline data points divided by 25. However, calculating the average of the baseline data points is only one example of determining a resting HR using identified baseline data points. Various other algorithms may be used.

Baseline and non-baseline data points (or segments) may be identified in various manners. In some embodiments, the analysis module 115 may use the first derivative waveform 204 to label or assign a status to one or more data points in the HR waveform 202 (or HR signal). For example, the analysis module 115 may determine one or more designated values (e.g., a threshold value or a minimum value) or a designated range between two values. The HR data points may then be labeled as baseline or non-baseline data points based on whether the derivative data points that correspond to the HR data points exceed a designated value, do not exceed a designated value, or are within a designated range of values.

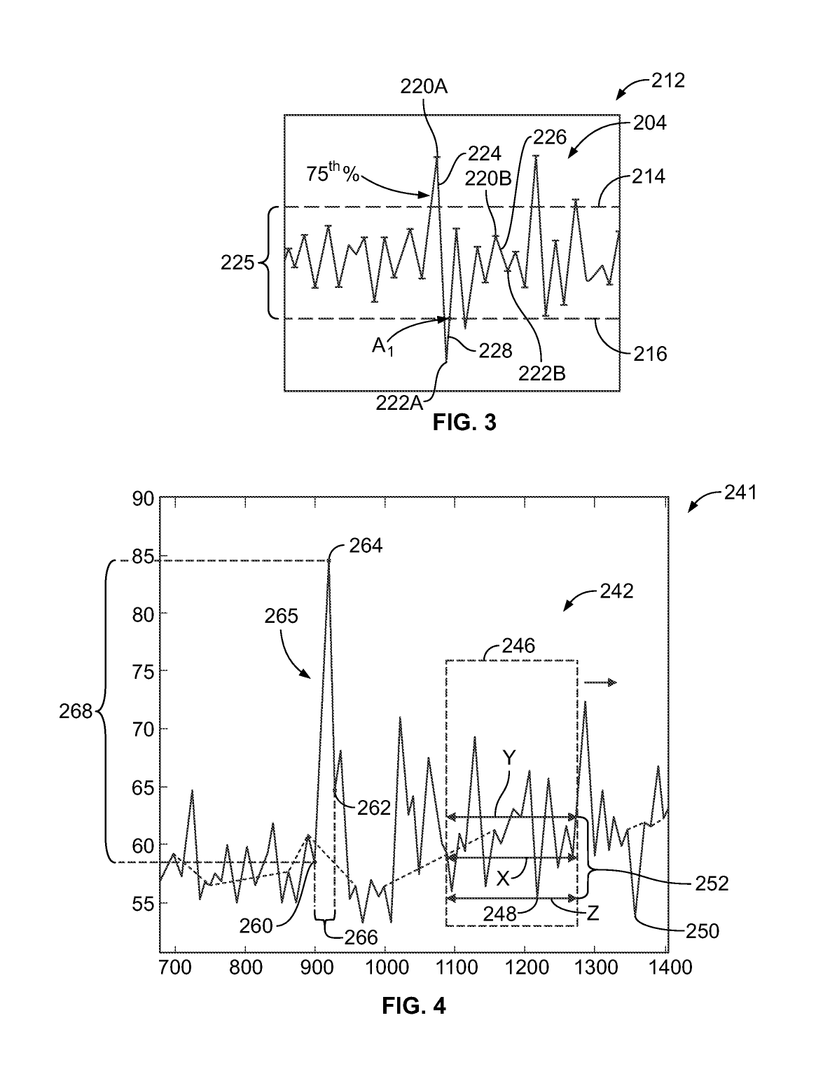

FIG. 3 illustrates a portion of the first derivative waveform 204 in the time window 212 that extends between about t=500 seconds to about t=800 seconds. In one or more embodiments, the analysis module 115 (FIG. 1A) is configured to identify data points or segments of the first derivative waveform 204 that correspond to substantial changes in the HR signal (FIG. 2), which may also be referred to as spikes, substantial deviations, substantial aberrations, and the like.

To this end, the analysis module 115 may determine a threshold value 214 that is to be applied to the first derivative waveform 204. The threshold value 214 may be determined in various manners. For example, the analysis module 115 may utilize a predetermined threshold value, such as a number between 0.5 and 1.0. The analysis module 115 may also analyze the data points in the time window 212 and identify a median data point of all the positive data points in the first derivative waveform 204. In some embodiments, the analysis module 115 may analyze the data points of the first derivative waveform 204 in the time window 212 and set the threshold value 214 at a designated percentile. For example, the threshold value 214 may be set at the 75th percentile of the data points within the time window 212 as shown in FIG. 3.

Accordingly, the HR data points that correspond to derivative data points that do not exceed the threshold value 214 may be identified as baseline data points and may be considered in the calculation of the resting HR for the time period of 500 seconds<t<800 seconds. The HR data points that correspond to derivative data points that exceed the threshold value 214 may be identified as non-baseline data points. The non-baseline data points may not be considered in the calculation of the resting HR. Accordingly, abnormally elevated values in the HR signal (i.e., spikes in the HR signal) may be removed from the calculation of the resting HR by identifying substantial changes in the HR signal using the first derivative waveform 204.

In some embodiments, the analysis module 115 may also determine a minimum designated value 216. In a similar manner as described above, the HR data points that correspond to derivative data points that are below the designated value 216 may be identified as non-baseline data points. The HR data points that correspond to derivative data points that exceed the designated value 216 may be identified as baseline data points. In some embodiments, the baseline data points correspond to the derivative data points that exceed the designated value 216 but are also below the threshold value 214.

In some embodiments, the analysis module 115 may identify peaks 220 and troughs 222 of the first derivative waveform 204. Peaks 220A, 220B and troughs 222A, 222B are shown in FIG. 3. The peaks 220 and troughs 222 may be identified by analyzing the first derivative waveform 204 or by obtaining a second derivative waveform (not shown) to identify where the first derivative waveform 204 changes in slope. In such embodiments, the analysis module 115 may identify derivative data points of the first derivative waveform 204 that are located within a designated range 225 between, for example, values 214, 216.

As another example, FIG. 3 shows a derivative segment 224 that extends between the peak 220A and the trough 222A. FIG. 3 also shows a derivative segment 226 that extends between the peak 220B and the trough 222B. The derivative segment 224 extends beyond each of the values 214, 216. The derivative segment 224 may represent a time in which the HR signal changes substantially. For example, the HR signal was rising rapidly at peak 220A and falling rapidly at trough 222A. More specifically, the derivative segment 224 includes data points that have values that exceed 2 and data points that have values that fall below −2. In such embodiments, the HR signal may be characterized as changing substantially (or spiking).

Unlike the derivative segment 224, the derivative segment 226 is located entirely between the values 214, 216. The derivative segment 226 may represent a time in which the HR signal does not change substantially. In other words, the derivative segment 226 and similar segments correspond to time periods in which the individual has a substantially steady HR. The analysis module 115 may identify such derivative segments in the time window that correspond to a steady HR. The HR data points that correspond to the derivative data points of the steady HR may be identified as baseline data points and a resting HR may be calculated therefrom.

Various modifications may be made to the above-described algorithms. For example, in some embodiments, the analysis module 115 may use derivative segments of the first derivative waveform 204 that are entirely within the designated range 225 and derivative segments that enter the designated range 225 with a positive slope. For example, a derivative segment 228 extends from the trough 222A. The derivative segment 228 enters the designated range 225 with a positive slope at point A₁. The derivative segment 228 may represent a time after the HR signal has fallen rapidly and is beginning to return to a steady state. In such embodiments, the analysis module 115 may consider the HR data points corresponding to the derivative data points that are part of the derivative segment 228 and that are within the designated range 225. The derivative segment 224 (or portions thereof) may not be considered by the analysis module 115 because the derivative segment 224 enters the designated range 225 with a negative slope. However, in other embodiments, the analysis module 115 may consider at least a portion of the derivative segment 224.

In some embodiments, the analysis module 115 calculates a resting HR for separate time windows. For example, the analysis module 115 may calculate a resting HR for separate 30 second blocks of time (e.g., for a time window 0 seconds<t<30 seconds, a time window 30 seconds<t<60 seconds, and a time window 60 seconds<t<90 seconds). However, in other embodiments, the time window may shift (or slide) along the time axis in steps that are less than the length of the time window (e.g., in 5 second steps or the period of the current heart beat) and the analysis module 115 may continuously re-calculate (e.g., update) the resting HR. More specifically, as the time window shifts along the time axis, new data points are added to the time window and older data points are removed from the time window. The analysis module 115 may re-calculate the resting HR for every new data point that enters the shifting time window or for every new set of data points (e.g., every new ten data points) that enters the shifting time window.

In one or more embodiments, the analysis module 115 may generate a baseline trend that may be based on the baseline data points and may represent the resting HR as a continuous waveform over an extended period of time. For instance, because the resting HR may differ between adjacent time windows or may differ as the resting HR of the shifting time window is updated, the baseline trend changes over time. More specifically, the resting HR may be a dynamic parameter that changes over an extended period of time.

For instance, returning to FIG. 2, a baseline trend 230 (indicated as a bolded waveform) is overlaid with the HR waveform 202. The baseline trend 230 is calculated using the shifting time window. As shown in FIG. 2, a slope of the baseline trend 230 near the waveform spikes 232-234 does not change or does not change as significantly as the HR waveform 202. For the waveform spikes 232-234, the analysis module 115 may not consider (i.e., may exclude) the HR data points associated with the HR signal. In embodiments that utilize the shifting time window, the resting HR of the time window may be biased toward the resting HR that was previously calculated. As such, the baseline trend may be smoother than other embodiments that do not use the shifting time window.

In embodiments that utilize the shifting time window, the threshold values 214, 216 discussed above may also be updated as new data points are added to the time window and as older data points are removed. For instance, when the threshold value 214 is a percentile of the data points in the time window 212, the threshold value may adjust in accordance with the modified set of data points.

Baseline and non-baseline data points may be identified in other manners than as described above. For example, FIG. 4 illustrates a HR signal graph 241 that includes an HR waveform 242 showing an HR signal over time. In some embodiments, the analysis module 115 may identify a lowest HR value in a shifting time window 246. By way of example, the lowest HR value in the time window 246 shown in FIG. 4 corresponds to data point 248. However, as the time window 246 shifts to the right, the data point 248 may be removed from the time window 246 and/or a new lower data point 250 may enter the time window as a new data point. When either occurs, the resting HR of the time window 246 may change. Accordingly, the resting HR may be the lowest HR value in a shifting time window. In other embodiments that utilize separate time windows, the resting HR may be the lowest HR value in each separate time window.

In some embodiments, a percentile filtering method may be used. More specifically, the resting HR for the shifting time window 246 may be based on a designated percentile of the data points in the time window 246. The analysis module 115 may determine a HR value of the lower Xth percentile by determining a difference between a largest HR value and a lowest HR value in the shifting time window 246. If the Xth percentile corresponds to a 20th percentile, then the analysis module 115 may determine 20% of the difference and add that amount to the lowest HR value. The analysis module 115 may then identify the data points that have HR values that are less than or equal to the Xth percentile.

By way of example, the highest HR value in the time window 246 shown in FIG. 4 is about 68 heart beats/minute and the lowest HR value is about 56 heart beats/minute. Based on this data, the 20th percentile is about 58 heart beats/minute. The data points that have HR values less than 58 heart beats/minute may be characterized as baseline data points. The baseline data points may be averaged to determine the resting HR for the time window 246. Again, as the time window 246 shifts to the right, the highest and/or lowest HR values may change thereby changing the HR value of the Xth percentile.

In some embodiments, the resting HR may be defined as the Xth percentile in a data set. For example, if the HR values in the data set (e.g., the HR values corresponding to the data points in the time window 246) are ordered in a range from lowest to highest value, the Xth percentile exists at X percent from the lowest value of the ordered range. For example, if there are one hundred HR values in the time window 246, the 50th percentile (e.g., the median) is the 50th HR value from the least HR value in the ordered range and the 20th percentile is the 20th HR value from the least HR value.

Other percentile filtering methods may be used. For example, as shown in FIG. 4, a designated range 252 between two percentiles (Y and Z) may be identified by the analysis module 115. The Y and Z percentiles may be predetermined and are generally in the bottom half of the HR values in the time window 246. For example, the Z percentile may be about 0% and the Y percentile may be 30%. Another example is about 5% and 25% for Z and Y, respectively, or 5% and 15%, respectively. The data points that are located in the designated range 252 may be characterized as baseline data points. The baseline data points may be averaged to determine the resting HR for the time window 246.

In yet another example, the analysis module 115 may analyze a contour or shape of the HR waveform 242 to identify one or more spikes in the HR signal. The data points corresponding to the spikes may be characterized as non-baseline data points and/or the data points that do not correspond to the spikes may be characterized as baseline data points. As one example, the analysis module may identify adjacent troughs 260 and 262 of the HR waveform 242. Between the adjacent troughs 260, 262, the HR waveform 242 may have a peak 264. The analysis module 115 may determine dimensions of a waveform structure 265 that is defined by segments of the HR waveform 242 that extend between the adjacent troughs 260, 262 and the peak 264. For example, the analysis module 115 may calculate a width 266 measured along the time axis between the troughs 260, 262 and a height 268 measured along the HR axis between the lowest trough 260 and the peak 264. If a ratio of the height 268 to the width 266 exceeds a designated amount (e.g., 8:1, 10:1, or more), then the waveform structure 265 may be identified as a spike and the corresponding data points that are located between the troughs 260, 262 may be identified as non-baseline data points. The above is one example of waveform analysis that may be used to identify excessive spiking in the HR signal and other waveform analyses may be used.

The above-described methods of determining baseline and/or non-baseline data points are exemplary only and other methods may be used. Moreover, a combination of methods may be used. For example, the analysis module 115 may initially identify waveform structures that are suspected as being spikes. To determine if a waveform structure is a suspected spike, the analysis module 115 may apply a threshold value (e.g., 50th percentile of the time window or 75 heart beats/minute) to the HR waveform 242. If a data point that corresponds to a peak exceeds the threshold value, than the segments that extend from the peak to corresponding troughs may be identified as a suspected spike. Subsequent waveform analysis as described above may then be applied.

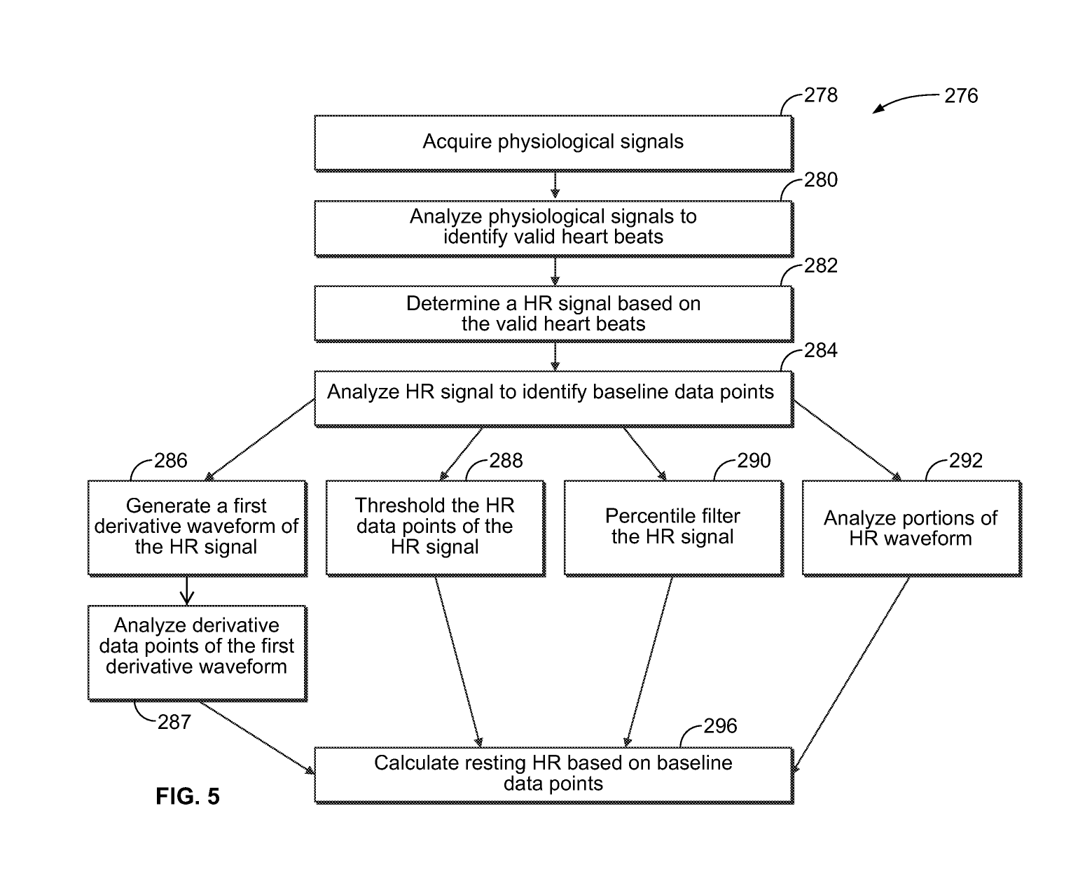

FIG. 5 illustrates a flow chart of a method 276 of determining a resting HR. The method may be performed by various systems, such as the system 100 or the PPG system 310 described below. The method 276 may include acquiring at 278 physiological signals. The physiological signals may be PPG signals, ECG signals, PCG signals, and/or ultrasound signals that characterize or describe cardiac activity. The physiological signals may be obtained from the individual for at least a designated period of time. At 280, the physiological signals may be analyzed to identify valid heart beats from the physiological signals. Various pre-processing protocols and algorithms may be used to validate heart beats in the physiological signals.

At 282, a HR signal based on the valid heart beats is determined. The HR signal may indicate the HR of the individual as a function of time. The HR signal includes a series of data points that are obtained over the designated period of time. The method 276 may also include analyzing at 284 the HR signal to identify baseline data points from the series of data points. At 296, a resting HR may be calculated that is based on the baseline data points.

The analyzing operation 284 may include analyzing and, optionally, processing the HR signal to identify the baseline data points. For example, the analyzing operation 284 may include generating at 286 a first derivative waveform of the HR signal and analyzing at 287 the derivative data points of the first derivative waveform to identify the baseline data points. The analyzing at 287 may include determining the derivative data points that exceed a predetermined threshold value, that do not exceed a predetermined threshold value, or that are located within a designated range of values. For the identified derivative data points, the corresponding HR data points (i.e., the data points of the HR signal) may be labeled as baseline data points. By way of one example, if the derivative data point at t=300 seconds has a derivative value that is identified as being located within a designated range of values, then the HR data point that at t=300 seconds is identified as a baseline data point and the HR value may be considered in calculating the resting HR.

The analyzing operation 284 may also include thresholding at 288 the HR data points to identify the baseline data points. The thresholding at 288 may include identifying the HR data points that do not exceed a predetermined threshold value and identifying such data points as baseline data points. The analyzing operation 284 may also include, at 290, percentile filtering the HR signal as described above. In some embodiments, the analyzing operation 284 may include analyzing at 292 one or more portions of a HR waveform that is based on the HR signal to identify one or more spikes. The data points that are not part of a spike may be labeled as baseline data points.

FIG. 6 illustrates an isometric view of a as PPG system 310, according to an embodiment. The PPG system 310 may be configured to, among other things, determine a resting HR of an individual. The PPG system 310 may be configured to analyze and/or process PPG signals as described above with respect to FIGS. 1-5 and the system 100. While the system 310 is shown and described as a PPG system 310, the system may be various other types of physiological detection systems, such as an ECG system, a PCG system, and the like.

The PPG system 310 may be a pulse oximetry system, for example. The system 310 may include a PPG sensor 312 and a PPG monitor 314. The PPG sensor 312 may include an emitter 316 configured to emit light into tissue of a patient. For example, the emitter 316 may be configured to emit light at two or more wavelengths into the tissue of the patient. The PPG sensor 312 may also include a detector 318 that is configured to detect the emitted light from the emitter 316 that emanates from the tissue after passing through the tissue. In other embodiments, the system 310 may include a plurality of sensors forming a sensor array in place of the PPG sensor 312. In such embodiments, the sensor array may include a complementary metal oxide semiconductor (CMOS) sensor, a charged coupled device (CCD) sensor, or a combination thereof.

The emitter 316 and the detector 318 may be configured to be located at opposite sides of a digit, such as a finger or toe, in which case the light that is emanating from the tissue passes completely through the digit. The emitter 316 and the detector 318 may be arranged so that light from the emitter 316 penetrates the tissue and is reflected by the tissue into the detector 318, such as a sensor designed to obtain pulse oximetry data.

The sensor 312 (or sensor array) may be operatively connected to and draw power from the monitor 314. Optionally, the sensor 312 may be wirelessly connected to the monitor 314 and include a battery or similar power supply (not shown). The monitor 314 may be similar to the monitor 106 described above and may be configured to analyze physiological signals and calculate physiological parameters based at least in part on data received from the sensor 312 relating to light emission and detection. Alternatively, the calculations may be performed by and within the sensor 312 and the result of the oximetry reading may be passed to the monitor 314. Additionally, the monitor 314 may include a display 320 configured to display the physiological parameters (e.g., current HR, resting HR, and other physiological information) or information about the system 310. The monitor 314 may also include a speaker 322 configured to provide an audible sound that may be used in various other embodiments, such as for example, sounding an audible alarm in the event that physiological parameters are outside a predefined normal range.

The system 310 may also include a multi-parameter workstation 326 operatively connected to the monitor 314. The workstation 326 may be or include a computing system 330, such as standard computer hardware. The computing system 330 may be similar to the monitor (or computing system) 106 described above and include one or more modules and control units, such as processing devices that may include one or more microprocessors, microcontrollers, integrated circuits, memory, such as read-only and/or random access memory, and the like. The workstation 326 may include a display 328, such as a cathode ray tube display, a flat panel display, such as a liquid crystal display (LCD), light-emitting diode (LED) display, a plasma display, a touch-sensitive display, or any other type of display. The computing system 330 of the workstation 326 may be configured to calculate physiological parameters and to show information from the monitor 314 and from other medical monitoring devices or systems (not shown) on the display 328. For example, the workstation 326 may be configured to display an estimate of a patient's blood oxygen saturation generated by the monitor 314 (referred to as an SpO₂ measurement), HR information from the monitor 314, and blood pressure from a blood pressure monitor (not shown) on the display 328.

The monitor 314 may be communicatively coupled to the workstation 326 via a cable 334 and/or 332 that is coupled to a sensor input port or a digital communications port, respectively and/or may communicate wirelessly with the workstation 326. Additionally, the monitor 314 and/or workstation 326 may be coupled to a network to enable the sharing of information with servers or other workstations. The monitor 314 may be powered by a battery or by a conventional power source such as a wall outlet.

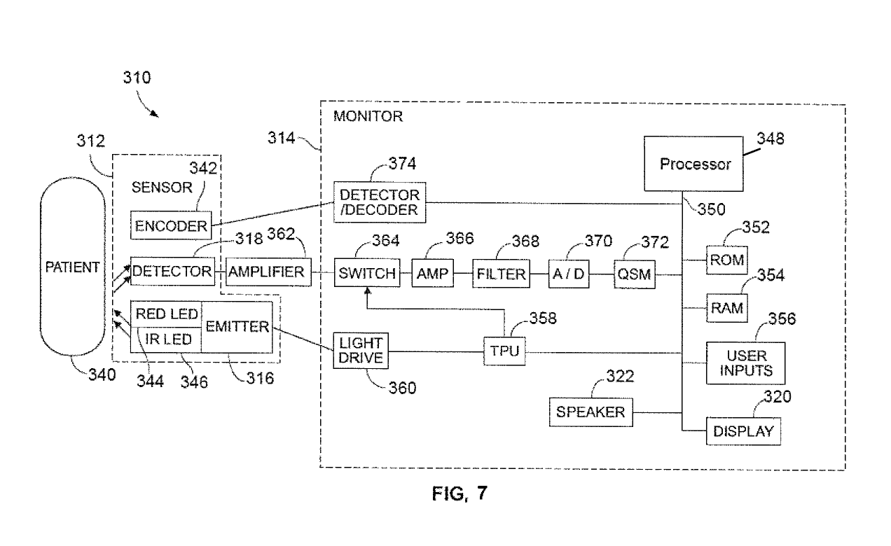

FIG. 7 illustrates a simplified block diagram of the PPG system 310, according to an embodiment. When the PPG system 310 is a pulse oximetry system, the emitter 316 may be configured to emit at least two wavelengths of light (for example, red and infrared) into tissue 340 of a patient. Accordingly, the emitter 316 may include a red light-emitting light source such as a red light-emitting diode (LED) 344 and an infrared light-emitting light source such as an infrared LED 346 for emitting light into the tissue 340 at the wavelengths used to calculate the patient's physiological parameters. For example, the red wavelength may be between about 600 nm and about 700 nm, and the infrared wavelength may be between about 800 nm and about 1000 nm. In embodiments where a sensor array is used in place of single sensor, each sensor may be configured to emit a single wavelength. For example, a first sensor may emit a red light while a second sensor may emit an infrared light.

As discussed above, the PPG system 310 is described in terms of a pulse oximetry system. However, the PPG system 310 may be various other types of systems. For example, the PPG system 310 may be configured to emit more or less than two wavelengths of light into the tissue 340 of the patient. Further, the PPG system 310 may be configured to emit wavelengths of light other than red and infrared into the tissue 340. As used herein, the term “light” may refer to energy produced by radiative sources and may include one or more of ultrasound, radio, microwave, millimeter wave, infrared, visible, ultraviolet, gamma ray or X-ray electromagnetic radiation. The light may also include any wavelength within the radio, microwave, infrared, visible, ultraviolet, or X-ray spectra, and that any suitable wavelength of electromagnetic radiation may be used with the system 310. The detector 318 may be configured to be specifically sensitive to the chosen targeted energy spectrum of the emitter 316.

The detector 318 may be configured to detect the intensity of light at the red and infrared wavelengths. Alternatively, each sensor in the array may be configured to detect an intensity of a single wavelength. In operation, light may enter the detector 318 after passing through the tissue 340. The detector 318 may convert the intensity of the received light into an electrical signal. The light intensity may be directly related to the absorbance and/or reflectance of light in the tissue 340. For example, when more light at a certain wavelength is absorbed or reflected, less light of that wavelength is received from the tissue by the detector 318. After converting the received light to an electrical signal, the detector 318 may send the signal to the monitor 314, which calculates physiological parameters based on the absorption of the red and infrared wavelengths in the tissue 340.

In an embodiment, an encoder 342 may store information about the sensor 312, such as sensor type (for example, whether the sensor is intended for placement on a forehead or digit) and the wavelengths of light emitted by the emitter 316. The stored information may be used by the monitor 314 to select appropriate algorithms, lookup tables and/or calibration coefficients stored in the monitor 314 for calculating physiological parameters of a patient. The encoder 342 may store or otherwise contain information specific to a patient, such as, for example, the patient's age, weight, and diagnosis. The information may allow the monitor 314 to determine, for example, patient-specific threshold ranges related to the patient's physiological parameter measurements, and to enable or disable additional physiological parameter algorithms. The encoder 342 may, for instance, be a coded resistor that stores values corresponding to the type of sensor 312 or the types of each sensor in the sensor array, the wavelengths of light emitted by emitter 316 on each sensor of the sensor array, and/or the patient's characteristics. Optionally, the encoder 342 may include a memory in which one or more of the following may be stored for communication to the monitor 314: the type of the sensor 312, the wavelengths of light emitted by emitter 316, the particular wavelength each sensor in the sensor array is monitoring, a signal threshold for each sensor in the sensor array, any other suitable information, or any combination thereof.

Signals from the detector 318 and the encoder 342 may be transmitted to the monitor 314. The monitor 314 may include a general-purpose control unit, such as a microprocessor 348 connected to an internal bus 350. The microprocessor 348 may be configured to execute software, which may include an operating system and one or more applications, as part of performing the functions described herein. A read-only memory (ROM) 352, a random access memory (RAM) 354, user inputs 356, the display 320, and the speaker 322 may also be operatively connected to the bus 350. The control unit and/or the microprocessor 348 (or other parts of the monitor 314) may include a pre-processing module, a validation module, a rate-determining module, an analysis module, and a GUI module, such as those described above.

The RAM 354 and the ROM 352 are illustrated by way of example, and not limitation. Any suitable computer-readable media may be used in the system for data storage. Computer-readable media are configured to store information that may be interpreted by the microprocessor 348. The information may be data or may take the form of computer-executable instructions, such as software applications, that cause the microprocessor to perform certain functions and/or computer-implemented methods. The computer-readable media may include computer storage media and communication media. The computer storage media may include volatile and non-volatile media, removable and non-removable media implemented in any method or technology for storage of information such as computer-readable instructions, data structures, program modules or other data. The computer storage media may include, but are not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which may be used to store desired information and that may be accessed by components of the system.

The monitor 314 may also include a time processing unit (TPU) 358 configured to provide timing control signals to a light drive circuitry 360, which may control when the emitter 316 is illuminated and multiplexed timing for the red LED 344 and the infrared LED 346. The TPU 358 may also control the gating-in of signals from the detector 318 through an amplifier 362 and a switching circuit 364. The signals are sampled at the proper time, depending upon which light source is illuminated. The received signal from the detector 318 may be passed through an amplifier 366, a low pass filter 368, and an analog-to-digital converter 370. The digital data may then be stored in a queued serial module (QSM) 372 (or buffer) for later downloading to RAM 354 as QSM 372 fills up. In an embodiment, there may be multiple separate parallel paths having amplifier 366, filter 368, and ND converter 370 for multiple light wavelengths or spectra received. In some embodiments, the amplifier 366, the low pass filter 368, and the analog-to-digital converter 370 are part of a pre-processing module.

The microprocessor 348 may be configured to determine the patient's physiological parameters, such as SpO₂ and pulse rate, using various algorithms and/or look-up tables based on the value(s) of the received signals and/or data corresponding to the light received by the detector 318. The signals corresponding to information about a patient, and regarding the intensity of light emanating from the tissue 340 over time, may be transmitted from the encoder 342 to a decoder 374. The transmitted signals may include, for example, encoded information relating to patient characteristics. The decoder 374 may translate the signals to enable the microprocessor 348 to determine the thresholds based on algorithms or look-up tables stored in the ROM 352. The user inputs 356 may be used to enter information about the patient, such as age, weight, height, diagnosis, medications, treatments, and so forth. The display 320 may show a list of values that may generally apply to the patient, such as, for example, age ranges or medication families, which the user may select using the user inputs 356.

As noted, the PPG system 310 may be a pulse oximetry system. A pulse oximeter is a medical device that may determine oxygen saturation of blood. The pulse oximeter may indirectly measure the oxygen saturation of a patient's blood (as opposed to measuring oxygen saturation directly by analyzing a blood sample taken from the patient) and changes in blood volume in the tissue. Ancillary to the blood oxygen saturation measurement, pulse oximeters may also be used to measure the pulse rate (or HR) of a patient as described above. Pulse oximeters typically measure and display various blood flow characteristics including, but not limited to, the oxygen saturation of hemoglobin in arterial blood.

A pulse oximeter may include a light sensor, similar to the sensor 312, that is placed at a site on a patient, typically a fingertip, toe, forehead or earlobe, or in the case of a neonate, across a foot. The pulse oximeter may pass light using a light source through blood perfused tissue and photoelectrically sense the absorption of light in the tissue. For example, the pulse oximeter may measure the intensity of light that is received at the light sensor as a function of time. A signal representing light intensity versus time or a mathematical manipulation of this signal (for example, a scaled version thereof, a log taken thereof, a scaled version of a log taken thereof, and/or the like) may be referred to as the photoplethysmograph (PPG) signal. In addition, the term “PPG signal,” as used herein, may also refer to an absorption signal (for example, representing the amount of light absorbed by the tissue) or any suitable mathematical manipulation thereof. The light intensity or the amount of light absorbed may then be used to calculate the amount of the blood constituent (for example, oxyhemoglobin) being measured as well as the pulse rate and when each individual pulse occurs.

The light passed through the tissue is selected to be of one or more wavelengths that are absorbed by the blood in an amount representative of the amount of the blood constituent present in the blood. The amount of light passed through the tissue varies in accordance with the changing amount of blood constituent in the tissue and the related light absorption. Red and infrared wavelengths may be used because it has been observed that highly oxygenated blood will absorb relatively less red light and more infrared light than blood with lower oxygen saturation. By comparing the intensities of two wavelengths at different points in the pulse cycle, it is possible to estimate the blood oxygen saturation of hemoglobin in arterial blood.

The PPG system 310 and pulse oximetry are further described in United States Patent Application Publication No. 2012/0053433, entitled “System and Method to Determine SpO₂ Variability and Additional Physiological Parameters to Detect Patient Status,” United States Patent Application Publication No. 2010/0324827, entitled “Fluid Responsiveness Measure,” and United States Patent Application Publication No. 2009/0326353, entitled “Processing and Detecting Baseline Changes in Signals,” all of which are hereby incorporated by reference in their entireties.

It will be understood that the present disclosure is applicable to any suitable physiological signals and that PPG signals are used for illustrative purposes. Those skilled in the art will recognize that the present disclosure has wide applicability to other signals including, but not limited to other physiological signals (for example, electrocardiogram, electroencephalogram, electrogastrogram, electromyogram, HR signals, pathological sounds, ultrasound, or any other suitable biosignal) and/or any other suitable signal, and/or any combination thereof.

Various embodiments described herein provide a tangible and non-transitory (for example, not an electric signal) machine-readable medium or media having instructions recorded thereon for a processor or computer to operate a system to perform one or more embodiments of methods described herein. The medium or media may be any type of CD-ROM, DVD, floppy disk, hard disk, optical disk, flash RAM drive, or other type of computer-readable medium or a combination thereof.

The various embodiments and/or components, for example, the control units, modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as a floppy disk drive, optical disk drive, and the like. The storage device may also be other similar means for loading computer programs or other instructions into the computer or processor.

As used herein, the term “computer,” “computing system,” or “module” may include any processor-based or microprocessor-based system including systems using microcontrollers, reduced instruction set computers (RISC), application specific integrated circuits (ASICs), logic circuits, and any other circuit or processor capable of executing the functions described herein. The above examples are exemplary only, and are thus not intended to limit in any way the definition and/or meaning of the term “computer” or “computing system”.

The computer, computing system, or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments of the subject matter described herein. The set of instructions may be in the form of a software program. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to user commands, or in response to results of previous processing, or in response to a request made by another processing machine.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

As discussed above, embodiments may provide a system and method of determining a resting HR through analysis of physiological signals in which the resting HR is less influenced by momentary rises in the HR. Embodiments may provide a system and method of calculating a resting HR based on PPG signals or calculating other physiological parameters in a more reliable manner. Such physiological parameters may include stroke volume, cardiac output, or respiratory effort.

It is to be understood that the above description is intended to be illustrative, and not restrictive. For example, the above-described embodiments (and/or aspects thereof) may be used in combination with each other. In addition, many modifications may be made to adapt a particular situation or material to the teachings without departing from its scope. While the dimensions, types of materials, and the like described herein are intended to define the parameters of the disclosure, they are by no means limiting and are exemplary embodiments. Many other embodiments will be apparent to those of skill in the art upon reviewing the above description. The scope of the disclosure should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means—plus-function format and are not intended to be interpreted based on 35 U.S.C. §112, sixth paragraph, unless and until such claim limitations expressly use the phrase “means for” followed by a statement of function void of further structure.

What is claimed is:

1. A system for determining a resting heart rate (HR) of an individual, the system comprising:
a monitor configured to be operatively connected to a sensor that obtains physiological signals from an individual, the monitor configured to receive the physiological signals from the sensor, the monitor including:
a validation module configured to analyze the physiological signals to identify valid heart beats from the physiological signals;
a rate-determining module configured to determine an HR signal that is based on the valid heart beats, the HR signal including a series of data points; and
an analysis module configured to analyze the HR signal and identify baseline data points from the series of data points, the analysis module configured to calculate the resting HR based on the baseline data points.

2. The system of claim 1, wherein the analysis module is configured to obtain a first derivative of the HR signal and establish a derivative threshold value, the first derivative including derivative data points, wherein the analysis module identifies derivative data points of the first derivative that are below the derivative threshold value, wherein the baseline data points of the HR signal correspond to the derivative data points identified by the analysis module.

3. The system of claim 1, wherein the analysis module is configured to obtain a first derivative of the HR signal and establish a designated range, the analysis module identifying derivative data points of the first derivative that are within the designated range, wherein the baseline data points of the HR signal correspond to the derivative data points identified by the analysis module.

4. The system of claim 1, wherein the baseline data points have HR values that are less than a designated HR threshold value.

5. The system of claim 4, wherein the baseline data points also have HR values that are above a minimum designated value.

6. The system of claim 1, wherein the HR signal forms a HR waveform, the analysis module analyzing a contour of the HR waveform to identify a spike in the HR waveform, the spike including non-baseline data points.

7. The system of claim 1, wherein the resting HR is a dynamic resting HR that changes over an extended period of time, the analysis module calculating a baseline trend, the baseline trend being based on the baseline data points and representing the resting HR as a continuous waveform over the extended period of time.

8. The system of claim 1, wherein the physiological signals comprise photoplethysmogram (PPG) signals and the monitor comprises a PPG monitor.

9. A method for determining a resting heart rate (HR) of an individual, the method comprising:
acquiring physiological signals of the individual from a sensor, the physiological signals being obtained from the individual;
analyzing the physiological signals to identify valid heart beats from the physiological signals;
determining an HR signal based on the valid heart beats, the HR signal including a series of data points;
analyzing the HR signal to identify baseline data points from the series of data points; and
calculating the resting HR based on the baseline data points.

10. The method of claim 9, further comprising calculating a first derivative of the HR signal and designating a derivative threshold value, the first derivative including derivative data points, wherein the analysis operation includes identifying derivative data points of the first derivative that are below the derivative threshold value, wherein the baseline data points of the HR signal correspond to the derivative data points identified by the analysis operation.

11. The method of claim 9, wherein the baseline data points have HR values that are less than a designated HR threshold value.

12. The method of claim 9, wherein the HR signal forms a HR waveform, the analysis module analyzing a contour of the HR waveform to identify a spike in the HR waveform, the spike including the non-baseline data points.

13. The method of claim 9, wherein the resting HR is a dynamic resting HR that changes over an extended period of time and wherein the analysis operation includes calculating a baseline trend, the baseline trend being based on the baseline data points and representing the resting HR as a continuous waveform over the extended period of time.

14. The method of claim 9, wherein the physiological signals include photoplethysmogram (PPG) signals, the method further comprising at least one of displaying the resting HR on a display.

15. A tangible and non-transitory computer readable medium that includes one or more sets of instructions configured to direct a monitor to:
acquire physiological signals of the individual from a sensor;
analyze the physiological signals to identify valid heart beats from the physiological signals;
determine an HR signal based on the valid heart beats, the HR signal including a series of data points;
analyze the HR signal to identify baseline data points from the series of data points; and
calculate the resting HR based on the baseline data points.

16. The computer readable medium of claim 15, further configured to calculate a first derivative of the HR signal and designate a derivative threshold value, the first derivative including derivative data points, wherein the computer readable medium is further configured to identify derivative data points of the first derivative that are below the derivative threshold value, wherein the baseline data points of the HR signal correspond to the identified derivative data points.

17. The computer readable medium of claim 15, wherein the baseline data points have HR values that are less than a designated HR threshold value.

18. The computer readable medium of claim 15, wherein the HR signal forms a HR waveform, the computer readable medium further configured to analyze a contour of the HR waveform to identify a spike in the HR waveform, the spike including non-baseline data points.

19. The computer readable medium of claim 15, further configured to calculate a baseline trend, the baseline trend being based on the baseline data points and representing the resting HR in a continuous waveform over an extended period of time.

20. The computer readable medium of claim 15, wherein the physiological signals include photoplethysmogram (PPG) signals.
